(19) United States
(12) Patent Application Publication
(10) Pub. No.: US 2022/0121283 A1
(43) Pub. Date: Apr. 21, 2022

(54) FINGER CLIP BIOMETRIC VIRTUAL REALITY CONTROLLERS

(71) Applicant: Hewlett-Packard Development Company, L.P., Spring, TX (US)

(72) Inventors: Mithra Vankipuram, Palo Alto, CA (US); Hiroshi Horii, Palo Alto, CA (US); Rafael Ballagas, Palo Alto, CA (US)

(73) Assignee: Hewlett-Packard Development Company, L.P., Spring, TX (US)

(21) Appl. No.: 17/415,774

(22) PCT Filed: Jun. 12, 2019
(86) PCT No.: PCT/US2019/036790

§ 371 Date: Jun. 18, 2021

(51) Int. Cl. G06F 3/01; A61B 5/024; A61B 5/0533; A61B 5/389; A61B 5/00

(52) U.S. Cl. CPC G06F 3/014; G06F 3/015; G06F 3/016; A61B 5/7455; A61B 5/0533; A61B 5/389; A61B 5/6826; A61B 5/02438

(57) ABSTRACT

In example implementations, an apparatus is provided. The apparatus includes a biometric sensor, a motion sensor, and a housing. The biometric sensor is to collect biometric data. The motion sensor is to detect movement of a finger of a user that is translated into motion or a control input in a virtual reality (VR) application. The housing is to enclose the biometric sensor and the motion sensor. The housing includes a mechanical coupling to attach to the finger of the user.

### BACKGROUND

Virtual reality (VR) applications provide new experiences for users. The VR applications can provide an artificial environment created via software. The VR systems may include a range of hardware. For example, the VR systems may include an entire enclosure where the user can feel movement as the enclosure moves, or a head mounted display that can be worn by the user.

### BRIEF DESCRIPTION OF THE DRAWINGS

FIG. 1 is a block diagram of an example VR system with finger clip biometric VR controllers of the present disclosure;

FIG. 2 is a block diagram of an example of the finger clip biometric VR controller of the present disclosure;

FIG. 3 illustrates a block diagram of another example of the finger clip biometric VR controller of the present disclosure;

FIG. 4 illustrates a block diagram of another example of the finger clip biometric VR controller of the present disclosure; and

FIG. 5 is a flow chart of an example method for adjusting a haptic feedback of the finger clip biometric VR controller based on biometric data of the present disclosure.

### DETAILED DESCRIPTION

Examples described herein provide a finger clip biometric VR controller and a method for adjusting a haptic feedback of the finger clip biometric VR controller based on biometric data. As discussed above, VR applications provide new experiences for users. Some VR systems include a head mounted display that can be used with gloves that provide controls. However, gloves may not provide optimal control because the hand sizes of different individuals may be different. Thus, if sensors and controls are placed in a glove, the glove may not operate properly in a VR environment if the glove does not properly fit a user's hand.

In addition, the use of gloves may increase costs as different size gloves are manufactured to try to accommodate different hand sizes. Gloves may also be uncomfortable for the user as gloves can make the user's hands hot or uncomfortably warm.

In addition, some VR applications may benefit from the use of biometric data. The biometric data may help the VR application make adjustments in real-time. In addition, the biometric data may allow the VR application to personalize the VR experience for each user.

Examples herein provide a finger clip biometric VR controller. The controller may be clipped onto the end of a user's finger or fingertip. The controller may include a biometric sensor that can collect biometric data and transmit the data to a VR system through a wired or wireless connection. The biometric data can be processed or analyzed to adjust a haptic feedback in the finger clip biometric VR controller. As a result, the user's VR experience can be personalized in real-time based on the user's biometric data that is collected via the finger clip biometric VR controller.

In addition, the finger clip biometric VR controller may provide controls for the VR application. For example, individual finger movements can be detected and various finger movements can be used as control inputs for the VR application. In addition, the finger clips may be designed to fit on any user's fingers, unlike a glove.

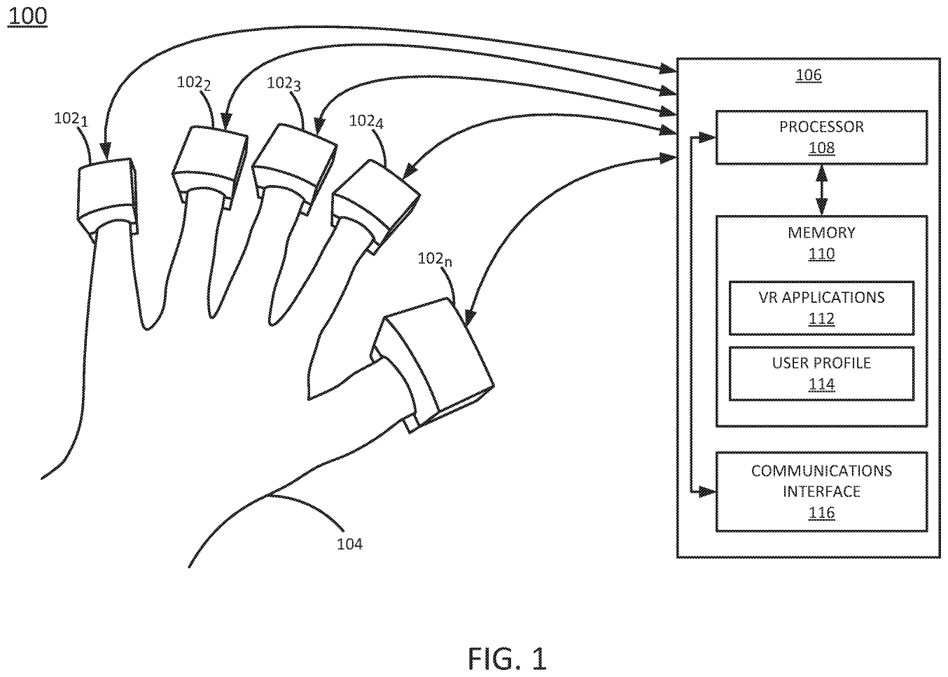

FIG. 1 illustrates an example VR system 100 of the present disclosure. The VR system 100 may include a VR controller 106 and finger clip biometric VR controllers 102₁-102ₙ (hereinafter also referred to individually as a finger clip biometric VR controller 102 or collectively as finger clip biometric VR controllers 102). Although a finger clip biometric VR controller 102 is illustrated as being clipped to the fingertip of each finger of a user's hand 104, it should be noted that any number of finger clip biometric VR controllers 102 may be deployed. For example, a single finger clip biometric VR controller 102 may be deployed, two finger clip biometric VR controllers 102 may be deployed (e.g., on an index finger and a thumb), and so forth.

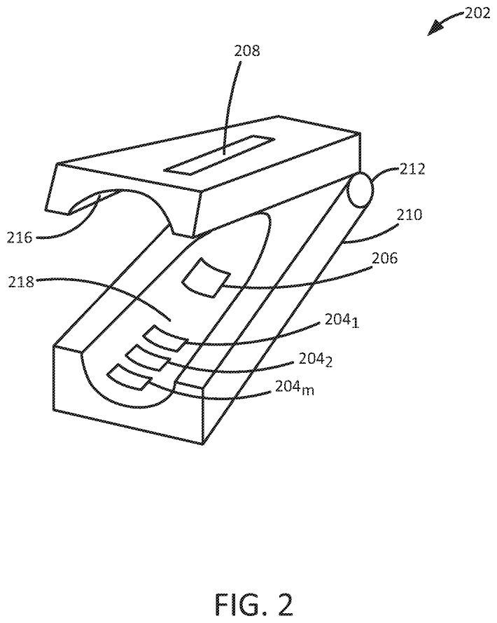

The finger clip biometric VR controllers 102 may be designed to clip onto the end of the fingers or the fingertips of the user's hand 104. In other words, the finger clip biometric VR controllers 102 are not a glove as used in other types of VR systems. As noted above, gloves can be cumbersome to wear, may not fit properly, may increase costs as different sized gloves are manufactured, and the like. The finger clip biometric VR controllers 102 may have a mechanical coupling to attach to the finger of a user's hand 104. The mechanical coupling may be a clam shell design that clips or mechanically clamps onto the fingertips of the user's hand 104. The design of the finger clip biometric VR controllers 102 is illustrated in FIGS. 2-4 and discussed in further details below.

In one example, the finger clip biometric VR controllers 102 may include biometric sensors that can collect biometric data of a user while the user is interacting with a VR application 112. The biometric data may include data such as heart rate, galvanic skin response (GSR), electromyography (EMG), and the like.

The finger clip biometric VR controllers 102 may include haptic feedback components, as discussed in further details below. The amount of haptic feedback and/or the type of haptic feedback provided to the user can be adjusted in real-time based on the biometric data of the user that is collected. The types of haptic feedback components may include components that can stretch the skin of the finger tips, provide vibration, provide texture via a surface with different nodes that can be individually controlled, provide pulsation, and the like.

In one example, the finger clip biometric VR controllers 102 may be communicatively coupled to the VR controller 106. For example, the finger clip biometric VR controllers 102 may be connected via a wired connection or a wireless connection. In one example, the finger clip biometric VR controllers 106 may communicate wirelessly via a Bluetooth connection. As a result, the biometric data measured by the finger clip biometric VR controllers 102 may be transmitted to the VR controller 106 for analysis and processing.

The biometric data can be analyzed by the VR controller 106 and the VR controller 106 may transmit a control signal back to the finger clip biometric VR controllers 102. The control signal may adjust the amount or type of haptic feedback that is provided by the finger clip biometric VR controllers 102.

In one example, the control signal may be unique for each finger clip biometric VR controller 102. For example, the adjustment to the amount and/or type of haptic feedback in the finger clip biometric VR controller 102₁ may be different than the adjustment to the amount and/or type of haptic feedback in the finger clip biometric VR controller 102₄. In another example, the control signal may make the same adjustment to the amount and/or type of haptic feedback in each one of the finger clip biometric VR controllers 102.

In one example, the VR controller 106 may include a processor 108, a memory 110, and a communications interface 116. It should be noted that the VR controller 106 has been simplified for ease of explanation and may include additional components that are not shown. For example, the VR controller 106 may be deployed as a head mounted display that includes a display, speakers to provide audio, a microphone to receive audio input, and the like.

In one example, the processor 108 may be communicatively coupled to the memory 110 and to the communications interface 116. The memory 110 may be any type of non-transitory computer readable medium such as a hard disk drive, random access memory (RAM), read only memory (ROM), and the like. The memory 110 may include VR applications 112 and a user profile 114.

In one example, the VR applications 112 may include instructions executed by the processor 108 to generate a virtual reality environment. The user may interact with the VR environment with the finger clip biometric VR controllers 102. The finger clip biometric VR controllers 102 may provide control inputs to execute instructions in the VR application 112 and movement information to move corresponding fingers or appendages in the VR application 112.

In one example, the user profile 114 may include information about a user and baseline biometric information about the user. In one example, the information about the user may include name, age, weight, height, user preferences, user settings for the VR application 112, and so forth.

In one example, the baseline biometric information about the user may be collected during an initialization process or a testing application. For example, the user may interact with the testing application and the biometric data may be measured. The testing application may determine a baseline level for the biometric data. The user may be asked questions during the testing application to determine whether certain amounts of haptic feedback or certain types of haptic feedback are too little or too much. Based on the user responses and the biometric data that is collected during the testing application, a baseline or a threshold biometric data associated with the user may be stored in the user profile 114.

When the user interacts with the VR application 112 after the testing application, the biometric data may be collected by the finger clip biometric VR controllers 102. In one example, the biometric data may be averaged from the finger clip biometric VR controllers 102 when more than one finger clip biometric VR controller 102 is deployed. The biometric data may be compared to the thresholds for the biometric data of the user. If the biometric data is above the threshold (e.g., the haptic feedback is too intense), the VR controller 106 may send a control signal to the finger clip biometric VR controllers 102 to reduce the amount or type of haptic feedback. If the biometric data is below the threshold (e.g., the haptic feedback is not enough or not stimulating the user), the VR controller 106 may send a control signal to the finger clip biometric VR controllers 102 to increase the amount or type of haptic feedback.

In one example, the communications interface 116 may be a wired or wireless communication interface. The communications interface 116 may establish a two-way communication path with the finger clip biometric VR controllers 102. In one example, the communications interface 116 may be a wireless radio that can communication over a wireless communication protocol, such as Bluetooth.

FIG. 2 illustrates an example of a finger clip biometric VR controller 202. In one example, the finger clip biometric VR controller 202 may include a housing 210 that has a clam shell design. For example, the housing 210 may include two halves that are coupled via a spring loaded hinge 212. Each half of the housing 210 may include a curved portion 216 and 218 that can fit around the fingertip of the user's hand 104. The housing 210 may be fabricated from plastic or metal.

In one example, the finger clip biometric VR controller 202 may include sensors 204₁-204ₘ (also referred to herein individually as a sensor 204 or collectively as sensors 204) to collect and/or measure biometric data from the fingertip of the user's hand 104. In one example, one sensor 204 may be a heart rate sensor, another sensor 204 may be a GSR sensor, another sensor 204 may be an EMG sensor, and so forth.

In one example, the finger clip biometric VR controller 202 may include a motion sensor 206. The motion sensor 206 may be a gyroscope or any other motion tracking device that can track the movement of the finger clip biometric VR controller 202.

In one example, the finger clip biometric VR controller 202 may include a wireless transmitter 208. The wireless transmitter 208 may include a wireless radio that can communicate using wireless communication protocols, such as Bluetooth.

In one example, the finger clip biometric VR controller 202 may also include additional components that are not shown. For example, the finger clip biometric VR controller 202 may include a processor, a memory to temporarily store the biometric data that is measured, a battery to power the finger clip biometric VR controller 202, and the like. It should be noted that the sensors 204 and 206 and the wireless transmitter 208 may be located or arranged in any desired location in the housing 210 even though the sensors 204 and 206 and the wireless transmitter 208 are illustrated in a particular location and arrangement in FIG. 2.

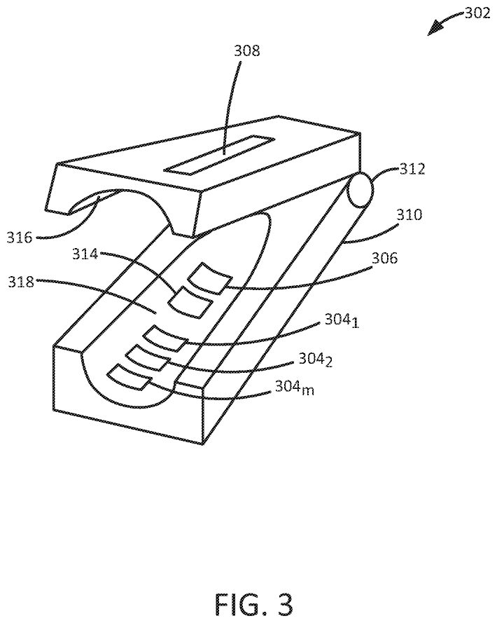

FIG. 3 illustrates an example of a finger clip biometric VR controller 302. In one example, the finger clip biometric VR controller 302 may include a housing 310 that has a clam shell design. For example, the housing 310 may include two halves that are coupled via a spring loaded hinge 312. Each half of the housing 310 may include a curved portion 316 and 318 that can fit around the fingertip of the user's hand 104. The housing 310 may be fabricated from plastic or metal.

In one example, the finger clip biometric VR controller 302 may include sensors 304₁-304ₘ (also referred to herein individually as a sensor 304 or collectively as sensors 304) to collect and/or measure biometric data from the fingertip of the user's hand 104. In one example, one sensor 304 may be a heart rate sensor, another sensor 304 may be a GSR sensor, another sensor 304 may be an EMG sensor, and so forth.

In one example, the finger clip biometric VR controller 302 may include a motion sensor 306. The motion sensor 306 may be a gyroscope or any other motion tracking device that can track the movement of the finger clip biometric VR controller 302.

In one example, the finger clip biometric VR controller 302 may include a wireless transmitter 308. The wireless transmitter 308 may include a wireless radio that can communicate using wireless communication protocols, such as Bluetooth.

In one example, the finger clip biometric VR controller 302 may include a haptic feedback device 314. Although a single haptic feedback device 314 is illustrated in FIG. 3 it should be noted that a plurality of different haptic feedback devices 314 may be deployed for different types of haptic feedback. For example, the haptic feedback device 314 may provide haptic feedback such as stretching, vibrations, textures, pulsations, and the like.

In one example, the finger clip biometric VR controller 302 may also include additional components that are not shown. For example, the finger clip biometric VR controller 302 may include a processor, a memory to temporarily store the biometric data that is measured, a battery to power the finger clip biometric VR controller 302, and the like. It should be noted that the sensors 304 and 306, the wireless transmitter 308, and the haptic feedback device 314 may be located or arranged in any desired location in the housing 310 even though the sensors 304 and 306, the wireless transmitter 308, and the haptic feedback device 314 are illustrated in a particular location and arrangement in FIG. 3.

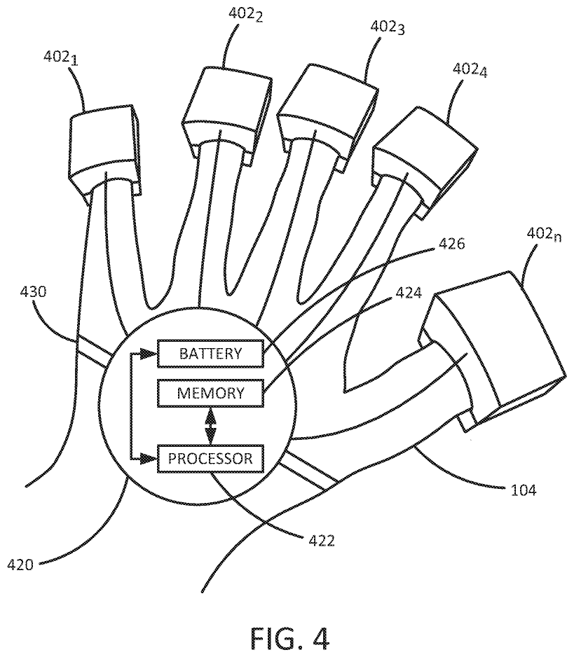

FIG. 4 illustrates a block diagram of another example of finger clip biometric VR controllers 402₁ to 402ₙ (also referred to herein individually as a finger clip biometric VR controller 402 or collectively as finger clip biometric VR controllers 402). In one example, the finger clip biometric VR controllers 402 may be similar to the finger clip biometric VR controllers 202 and/or 302 in that the finger clip biometric VR controllers 402 include a biometric sensor, a wireless transmitter and/or a haptic feedback device.

In one example, the finger clip biometric VR controllers 402 may be coupled to a body 420. The body 420 may enclose or house components that can be worn on the back of the user's hand 104. In one example, a strap 430 may be coupled to the body 420 to secure the body 420 to the user's hand 104.

In one example, the body 420 may include a processor 422, a memory 424, and a battery 426. The processor 422 may be communicatively coupled to the memory 424 and the battery 426. As a result, the finger clip biometric VR controllers 402 may be manufactured at a lower cost and some of the components can be moved into the body 420 and shared between the finger clip biometric VR controllers 402. This may make the finger clip biometric VR controllers 402 lighter and more comfortable when clipped onto the fingertips of the user's hand 104.

In one example, other components may also be moved to the body 420. For example, the wireless transmitter may also be moved to the body 420. Thus, each finger clip biometric VR controller 402 may transmit data to the body 420 and the body 420 may wirelessly transmit the biometric data to the VR controller 106.

In one example, the memory 424 may be any type of non-transitory computer readable medium. For example, the memory 424 may be a hard disk drive, RAM, ROM, and the like. In one example, the battery 426 may provide power to each one of the finger clip biometric VR controllers 402. The body 420 may include ports that are not shown. For example, the ports may include a power port to recharge the battery 426, a port to provide a wired connection to upload the biometric data from the memory 424, and the like.

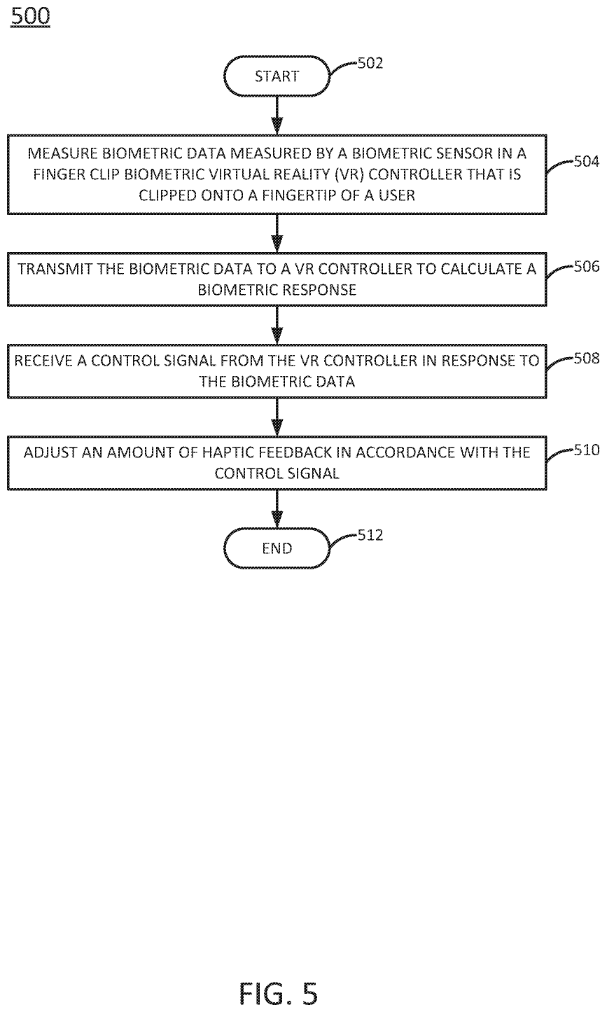

FIG. 5 illustrates a detailed flow chart of a method for adjusting a haptic feedback of a finger clip biometric VR controller based on biometric data of the present disclosure. In an example, the method 500 may be performed by the VR system illustrated in FIG. 1 or the finger clip biometric VR controller illustrated in FIGS. 2-4.

At block 502, the method 500 begins. At block 504, the method 500 measures biometric data measured by a biometric sensor in a finger clip biometric virtual reality (VR) controller that is clipped onto a fingertip of a user. The finger clip biometric VR controller may have a clam shell design that can be mechanically clipped onto the fingertip of the user, as noted above. The finger clip biometric VR controller may include at least one biometric sensor to measure biometric data, such as heart rate, GSR, EMG, and the like.

The biometric data may be measured while the user is interacting with a VR application. The finger clip biometric VR controller may measure the biometric data of the user continuously as the user is interacting with the VR application or may measure the biometric data periodically (e.g., every 10 seconds, every 30 seconds, every minute, and the like).

At block 506, the method 500 transmits the biometric data to a VR controller to calculate a biometric response. In one example, the biometric data may be sent continuously to the VR controller. For example, a two-way communication path may be established and maintained between the finger clip biometric VR controller and the VR controller while the user is interacting with the VR application. The biometric data may be sent in real-time to the VR controller for processing and/or analysis.

In one example the biometric data may be transmitted periodically. For example, the biometric data may be temporarily stored in a memory of the finger clip biometric VR controller. The biometric data may then be transmitted to the VR controller every few seconds or according to any other desired time period. The memory may be a circular buffer such that the older data can be overwritten as new biometric data is measured and stored.

In one example, the VR controller may calculate a biometric response. The biometric response may be an overall biometric value or level based on the different biometric parameters that are measured. For example, if heart rate, GSR, and EMG are measured, each biometric parameter may be correlated to a value. For example, a heart rate in the range of 65-75 beats per minute may be assigned a middle value of 5 on a scale of 1-10. Higher heart rates may be assigned a higher value and lower heart rates may be assigned a lower value.

The values may be weighted (e.g., heart rate may be more heavily weighted than GSR and EMG). In another example, each value may have an equal weight.

The calculated biometric response may be measured against a baseline or threshold of the user. The threshold may be stored in a user profile of the user in the VR controller, as discussed above. In one example, the baseline or threshold for the biometric response may be measured via the finger clip biometric VR controller during a testing application. Initial settings for the finger clip biometric VR controller may be set based on the biometric data that is collected during the testing application.

In one example, the initial settings may be set for a particular type of haptic feedback and an amount of each type of haptic feedback that is enabled. For example, based on the testing application, the finger clip biometric VR controller may be initially set to allow all types of haptic feedback at a medium setting. For example, the finger clip biometric VR controller may allow haptic feedback such as stretching, vibration, texture, and pulsating at a medium level. Based on the calculated biometric response compared to the threshold, the type and/or amount of haptic feedback may be adjusted with a control signal from the VR controller.

At block 508, the method 500 receives a control signal from the VR controller in response to the biometric data. For example, the control signal may control the type and/or amount of haptic feedback provided by the finger clip biometric VR controller. For example, the control signal may increase the amount of haptic feedback when the biometric response is below the threshold or may decrease the amount of haptic feedback when the biometric response is above the threshold.

At block 510, the method 500 adjusts an amount of haptic feedback in accordance with the control signal. For example, the biometric response may indicate that the user is overstimulated (e.g., the biometric response is greater than the threshold). The control signal may adjust the amount of haptic feedback to a low setting. In one example, the amount may be adjusted by reducing the amount of haptic feedback in each one of a plurality of finger clip biometric VR controllers or may be adjusted by reducing some of the finger clip biometric VR controllers.

In one example, the control signal may adjust a type of haptic feedback that is enabled. For example, the biometric data may show that the user is overstimulated by haptic feedback that provides texture. For example, certain portions of the VR application may call for texture haptic feedback and the biometric data may show spikes during these interactions. As a result, the control signal may disable texture haptic feedback in the finger clip biometric VR controller, while allowing the other types of haptic feedback to remain enabled.

In one example, the movements of the finger of the user may also be tracked by the finger clip biometric VR controller. The movements of the finger may be tracked and transmitted to the VR controller. The movements may be used to control a corresponding VR finger or appendage in the VR application in accordance with the movement of the finger. The finger movements may also be tracked to detect control inputs. For example, the user may swipe, pinch, scroll, and the like using finger movements, such as moving the fingertip left and right, pinching an index finger and a thumb, moving the fingertip up and down, and the like.

It should be noted that the method 500 may be repeated continuously while the user is interacting with the VR application. For example, the amount of haptic feedback may be initially decreased based on the measured biometric data as the user interacts with the VR application. However, over time the user may become comfortable with the VR application and the amount of haptic feedback may be gradually increased based on the measured biometric data.

At block 512, the method 500 ends. For example, the VR application may be terminated to end the method 500.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. An apparatus, comprising:
a biometric sensor to collect biometric data;
a motion sensor to detect movement of a finger of a user that is translated into motion or a control input in a virtual reality (VR) application; and
a housing to enclose the biometric sensor and the motion sensor, wherein the housing comprises a mechanical coupling to attach to the finger of the user.

2. The apparatus of claim 1, further comprising:
a wireless transmitter to transmit the biometric data to a VR controller.

3. The apparatus of claim 1, wherein a haptic feedback provided via the apparatus is adjusted based on the biometric data.

4. The apparatus of claim 1, wherein the mechanical coupling comprises a clam shell design with a spring loaded hinge.

5. The apparatus of claim 1, further comprising:
a body portion to enclose a battery, a processor, and a memory, wherein the body portion is coupled to the housing.

6. The apparatus of claim 1, wherein the biometric data comprises at least one of: heartrate, a galvanic skin response, or an electromyography.

7. An apparatus, comprising:
a biometric sensor to collect biometric data;
a motion sensor to detect movement of a finger of a user that is translated into motion or a control input in a virtual reality (VR) application;
a haptic feedback device to provide haptic feedback based on the biometric data; and
a housing to enclose the biometric sensor, the motion sensor, and the haptic feedback device, wherein the housing comprises a mechanical coupling to attach to the finger of the user.

8. The apparatus of claim 7, further comprising:
a wireless transmitter to transmit the biometric data to a VR controller.

9. The apparatus of claim 7, haptic feedback comprises stretching skin on the finger of the user, a vibration, a texture, or a pulse.

10. A method, comprising:
measuring, by a processor, biometric data measured by a biometric sensor in a finger clip biometric virtual reality (VR) controller that is clipped onto a fingertip of a user;
transmitting, by the processor, the biometric data to a VR controller to calculate a biometric response;
receiving, by the processor, a control signal from the VR controller in response to the biometric data; and
adjusting, by the processor, an amount of haptic feedback in accordance with the control signal.

11. The method of claim 10, comprising:
measuring, by the processor, the biometric data from the finger clip biometric VR controller during a testing application; and
setting, by the processor, an initial amount of haptic feedback based on the biometric data that is collected during the testing application.

12. The method of claim 10, wherein the measuring further comprises receiving biometric data from each fingertip of the user and the biometric response comprises an average of the biometric data from each fingertip of the user.

13. The method of claim 10, wherein the control signal is to increase the amount of haptic feedback based on the biometric data.

14. The method of claim 10, wherein the control signal is to decrease the amount of haptic feedback based on the biometric data.

15. The method of claim 10, further comprising:
tracking, by the processor, a movement of a finger; and
transmitting, by the processor, the movement of the finger to the VR controller to control a corresponding VR finger in a VR application in accordance with the movement of the finger.
